(19) United States
(12) Patent Application Publication
(10) Pub. No.: US 2007/0190047 A1
(43) Pub. Date: Aug. 16, 2007

(54) FORMULATIONS THAT INHIBIT PROTEIN AGGREGATION

(75) Inventors: Stephen R. Brych, Simi Valley, CA (US); Masazumi Matsumura, Thousand Oaks, CA (US)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: Amgen, Inc.

(21) Appl. No.: 11/461,333

(22) Filed: Jul. 31, 2006

Related U.S. Application Data
(60) Provisional application No. 60/703,547, filed on Jul. 29, 2005.
(60) Provisional application No. 60/703,551, filed on Jul. 29, 2005.

(51) Int. Cl. A61K 39/395

(52) U.S. Cl. 424/133.1

(57) ABSTRACT

Disclosed is a stable pharmaceutically acceptable formulation containing a pharmaceutically acceptable amount of a protein. Also disclosed are methods for preparing such formulations and methods for inhibiting protein aggregate formation induced by physical stresses associated with processing, manufacture, shipping, and storing protein formulations, particularly freeze/thaw stress.

This application claims priority to U.S. Provisional Patent Application Ser. No: 60/703,547, filed Jul. 29, 2005, and U.S. Provisional Patent Application Ser. No: 60/703,551, filed Jul. 29, 2005, each of which is incorporated herein by reference.

### FIELD OF THE INVENTION

The invention relates to pharmaceutical formulations containing a protein and to methods for making and using such formulations. More particularly, the invention relates to protein-containing pharmaceutical formulations that can inhibit formation of protein aggregate during manufacture and shipping. The invention also relates to methods for inhibiting formation of protein aggregate.

### BACKGROUND OF THE INVENTION

Proteins such as enzymes and antibodies, and protein fragments are unstable and susceptible to loss of activity and/or to formation of soluble or insoluble aggregates in aqueous solutions and when stored at low temperatures (i.e., at 0° C. or below). In the pharmaceutical industry, protein drug products are subjected to a number of stresses during manufacturing and shipping including, for example, purification procedures that involve harsh conditions (e.g., acid elution, heat, pH extremes, etc.); syringe manipulation, ultrafiltration, and diafiltration (high pressure and shear forces); agitation and freeze/thaw cycles. For extended storage, protein compositions (solutions/lyophilizates) are preferably frozen so that the protein is protected from degradation by slowing the kinetics of various degradation processes. This allows for retention of protein activity. However, some protein degradation can occur in the frozen state, usually due to ice-water/protein interface interactions and osmotic shock upon ice formation (Chang, et al., J. Pharm. Sci. 1996; 85(12):1325-1330; Carpenter and Crow, Cryobiology, 1988; 25:244-255). In particular repeated freeze/thaw cycles tend to increase protein aggregate formation, which can appear in solution making the solution appear cloudy (turbid). Another source of protein aggregation is agitation. In particular, during shipping a therapeutic protein, such as an antibody, is subject to agitation due to movement by surface and air transportation. During shipping proteins may interact with hydrophobic surfaces on a glass container or a plastic syringe as well as micro air bubbles in solution or air surface in a container. Such interactions of proteins with hydrophobic materials can induce protein aggregation. During the development, formulation, storage, and shipping of a therapeutic protein product, such as an antibody, suppression of insoluble aggregate formation is crucial for the retention of the drug substance because insoluble aggregate formation leads to unusable protein material.

Numerous processes and additives are known for the stabilization of proteins in solution. For example the stabilization of proteins by adding heat-shock proteins such as HSP25 is described in EP-A 0599344. Antibody stabilization by addition of block polymers composed of polyoxypropylene and polyoxyethylene in combination with phospholipids is described in EP-A 0318081. Immunoglobulins have been stabilized by adding a salt of a nitrogen-containing bases, such as arginine, guanidine, or imidazole. Other suitable additives for stabilization are polyethers (EP-A 0018609), glycerin, albumin and dextran sulfate (U.S. Pat. No. 4,808,705), detergents and surfactants such polysorbate-based surfactants (DE 2652636, GB 8514349), chaperones such as GroEL (Mendoza, J. A., Biotechnol. Tech., (10)1991 535-540), citrate buffer (WO 93/22335) or chelating agents (WO 91/15509). Although these additives enable proteins to be stabilized to some degree in solution, they suffer from certain disadvantages, for example, the necessity of additional processing steps for additive removal. Further, none of the processes described in the art is suitable for stabilizing proteins during repeated freezing and thawing processes such that no soluble or insoluble aggregates (or negligible amounts for therapeutic purposes) are formed during the manipulation (U.S. Pat. No. 6,238,664).

Freeze drying (lyophilization) is considered useful and effective for preservation of many biologically active materials, including proteins (Hershenson, U.S. Pat. No. 6,020,469). However, lyophilization induces its own stresses, including extreme concentration of the protein during the freezing process and removal of water, which may result in instability of the product. Hence, lyophilization may result in increased rates of crosslinking (covalent oligomer formation) and noncovalent aggregation, in addition to deamidation and oxidation, both of which can occur in the lyophilized state as well as the liquid state.

Thus, there remains a need in the art for protein formulations that have increased stability during processing, manufacturing, shipping, and storage. In particular, protein formulations that inhibit aggregate formation induced by one or more freeze/thaw cycles would be especially useful in the art.

### SUMMARY OF THE INVENTION

The invention relates to a protein formulation comprising a pharmaceutically acceptable amount of an antibody selected from antibody C, antibody D, antibody A, antibody B, and antibody E, or fragments thereof, in combination with an inhibitor of insoluble aggregate formation. In certain embodiments, the inhibitor of insoluble aggregate formation is MgCl₂, propylene glycol, Pluronic-F68, Poloxamer 188, ethanol, or combinations thereof A full description of antibodies A-E including how to make and use them can be found in U.S. Pat. Nos. and U.S. patent application Ser. Nos.: 10/180,648 (Antibody A); 10/891,658 (Antibody B); 5,789,554, 6,254,868, 09/038,955, 09/590,284, 10/153,882 (Antibody C); 60/638,961 (Antibody D); 6,235,883 (Antibody E) which are all incorporated herein by reference in their entirety, including the drawings.

The invention also relates to a protein formulation that inhibits formation of protein aggregate induced by one or more freeze/thaw cycles and by agitation, wherein the formulation comprises an inhibitor of insoluble aggregate formation. In certain embodiments, the inhibitor of insoluble aggregate formation is MgCl₂, propylene glycol, Pluronic-F68, Poloxamer 188, ethanol, or combinations thereof.

The invention relates to methods for inhibiting protein aggregate formation in a protein solution subject to one or more freeze/thaw cycles and agitation comprising: (a) selecting a buffer system, prior to the at least one freeze/thaw cycle or agitation; (b) contacting the buffer system of (a) with an amount of an inhibitor of insoluble aggregate formation effective to inhibit insoluble aggregate formation, prior to the at least one freeze/thaw cycle or agitation; and (c) contacting the buffer system and inhibitor of insoluble aggregate formation of (b), with an amount of a protein or protein fragment, prior to the at least one freeze/thaw cycle or agitation. In certain embodiments, the inhibitor of insoluble aggregate formation is MgCl₂, propylene glycol, Pluronic-F68, Poloxamer 188, ethanol, or combinations thereof

### DESCRIPTION OF THE DRAWINGS

FIG. 1 is a graph illustrating the dependence of cumulative total particle counts on pH, ranging from 4.0 to 8.0 in 5 mM K/PO4, 5 mM K/OAc buffer. The isoelectric point (pI) of each protein is: antibody E=6.5; antibody B=7.8; antibody D=8.1; antibody A=8.5; and antibody C=9.2.

FIG. 2 is a graph illustrating the dependence of cumulative total particle counts on MgCl₂ concentration for antibody E, over the same pH range as in FIG. 1. Data was collected for total formulation MgCl₂ concentrations of 0.0 mM, 30 mM, 100 mM, and 300 mM.

FIGS. 3A-3D are graphs illustrating the dependence of cumulative total particle counts on MgCl₂ concentration for antibody A, antibody B, antibody C, and antibody D, over the same pH range as in FIG. 1. Data was collected for each protein at total formulation MgCl₂ concentrations of 0.0 mM and 100 mM.

FIGS. 4A-4B are graphs illustrating the dependence of cumulative total particle counts on ethanol concentrations for antibody E. The buffer systems used for this data acquisition were 5 mM K/PO₄, 5 mM K/OAc, with or without 100 mM KCl or 100 mM NaCl (100 mM KCl, at pH 5.0 and 7.0; 100 mM NaCl, at pH 5 and 6). Ethanol concentrations ranged from 0-10% (v/v).

FIG. 5 is a graph illustrating the dependence of cumulative total particle counts on propylene glycol concentration for antibody E. The buffer systems used for this data acquisition were 5 mM K/PO₄, 5 mM K/OAc, with or without 100 mM KCl at pH 5.0 and 7.0. Propylene glycol concentrations ranged from 0-10% (v/v).

### DETAILED DESCRIPTION OF THE INVENTION

The invention provides protein formulations comprising an amount of at least one inhibitor of insoluble aggregate formation in an amount effective to inhibit the formation of insoluble aggregates in response to one or more freeze/thaw cycles, as well as methods for stabilizing a protein formulation against aggregate formation induced by one or more freeze/thaw cycles, methods for inhibiting protein aggregate formation in a protein solution that is subjected to one or more freeze/thaw cycles, methods for inhibiting protein aggregate formation induced by one or more freeze/thaw cycles, and methods for preparing a protein formulation stabilized against protein aggregate formation induced by one or more freeze/thaw cycles. Said methods have in common contacting a solution comprising a protein or a protein fragment with an amount of an inhibitor of insoluble aggregate formation effective to inhibit insoluble aggregate formation.

All references cited herein are incorporated by reference in their entirety, for all purposes.

As used herein, “inhibiting” protein aggregate formation means decreasing the amount of protein aggregate or preventing formation of additional protein aggregate in a protein-containing solution. Thus, inhibiting can encompass both decreasing and preventing the amount of protein aggregate in a protein formulation or solution. Decreasing or preventing is measured by comparing the amount of aggregate present in a protein-containing solution that comprises at least one inhibitor of insoluble aggregate formation with the amount of aggregate present in a protein-containing solution that does not comprise at least one inhibitor of insoluble aggregate formation.

As used herein, the terms “protein formulation” and “protein solution” are interchangeable. Further the term “protein” is understood within the sense of the invention as naturally occurring and recombinant proteins or protein fragments as well as chemically modified proteins and proteins containing amino acid substitutions and additions. Proteins which are stabilized for pharmaceutical compositions are preferably antibodies, antibody fusion proteins such as immunotoxins, enzymes and protein hormones such as erythropoietin, somatostatin, insulin, cytokines, interferons or plasminogen activators intended to encompass any amino acid sequence, particularly, polypeptides, peptides, enzymes, antibodies, and the like, and/or fragments thereof.

A “pharmaceutically effective amount” of protein or antibody refers to that amount which provides therapeutic effect in various administration regimens. Such amounts are readily determined by those skilled in the art. The amount of active ingredient will depend upon the severity of the condition being treated, the route of administration, etc. The compositions of the invention can be prepared containing amounts of protein of at least about 0.1 mg/mL, upwards of about 5 mg/mL. For the antibodies A-E, pharmaceutically effective amounts are preferably from about 0.1 mg/mL to about 20 mg/mL, or as disclosed in U.S. Pat. Nos. and U.S. patent application Ser. Nos.: 10/180,648 (Antibody A); 10/891,658 (Antibody B); 5,789,554, 6,254,868, 09/038,955, 09/590,284, 10/153,882 (Antibody C); 60/638,961 (Antibody D); 6,235,883 (Antibody E).

The term “Antibody A” is taken to mean the antibody disclosed in U.S. patent application Ser. No.: 10/180,648, or one or more fragments, mutations, deletions, additions, variants, truncations, or orthologs thereof.

The term “Antibody B” is taken to mean the antibody disclosed in U.S. patent application Ser. No.: 10/891,658, or one or more fragments, mutations, deletions, additions, variants, truncations, or orthologs thereof.

The term “Antibody C” is taken to mean the antibody disclosed in U.S. Pat. Nos. and patent application Ser. Nos.: 5,789,554, 6,254,868, 09/038,955, 09/590,284, 10/153,882, or one or more fragments, mutations, deletions, additions, variants, truncations or orthologs thereof The term “Antibody D” is taken to mean the antibody disclosed in U.S. patent application Ser. No.: 60/638,961, or one or more fragments, mutations, deletions, additions, variants, truncations, or orthologs thereof.

The term “Antibody E” is taken to mean the antibody disclosed in U.S. Pat. No.: 6,235,883 or one or more fragments, mutations, deletions, additions, variants, truncations, or orthologs thereof.

An “inhibitor of insoluble aggregate formation” is any compound or condition that can effectively inhibit the formation of protein aggregate in a solution comprising a protein or a protein fragment. In preferred embodiments, the inhibitor of insoluble aggregate formation is selected from pH; inorganic metal alkali and alkaline salts, such as MgCl₂ and the like; polyols, such as propylene glycol and the like; polymers, such as block polymers and block co-polymers (polyoxyethylene, polyoxypropylene, Pluronic-F68, Poloxamer 188, and the like); lower alcohols, such as ethanol, and the like; or combinations of two or more thereof.

In general, the formulations of the invention can contain other components in amounts preferably not detracting from the preparation of stable forms and in amounts suitable for effective, safe pharmaceutical administration.

In certain aspects the invention provides a formulation comprising a pharmaceutically acceptable amount of an antibody selected from the group consisting of antibody A, antibody B, antibody C, antibody D, antibody E, or fragments thereof, a buffer; and an inhibitor of insoluble aggregate formation.

In another aspect the invention provides a protein formulation having increased stability against insoluble aggregate formation induced by one or more freeze/thaw cycles, comprising a protein or protein fragment; an amount effective to inhibit insoluble aggregate formation of an inhibitor of insoluble aggregate formation; and a buffer system.

In another aspect the invention provides a protein formulation having increased stability against insoluble aggregate formation induced by agitation stress, comprising a protein or protein fragment; an amount effective to inhibit insoluble aggregate formation of an inhibitor of insoluble aggregate formation; and a buffer system. As used herein “agitation stress” is taken to mean any physical movement applied to the protein formulation either passively or actively. Non-limiting examples of agitation stresses, include bumping, dropping, shaking, swirling, vortexing, decanting, injecting, withdrawing (as into a syringe from a containing or vessel), and the like. The preferred protein formulation of the invention is particularly stabilized with respect to the forces of shipping and transportation.

In other aspects, the invention provides a protein formulation having increased stability against insoluble aggregate formation induced by one or more outside physical or chemical stresses, including non-limiting examples of heat stress, chemical stress (e.g., pH, low/high salt, and the like), fluid stress (e.g., compression stresses, such as those caused by fluid movement through constricted openings), and the like, comprising a protein or protein fragment; an amount effective to inhibit insoluble aggregate formation of an inhibitor of insoluble aggregate formation; and a buffer system.

In a preferred embodiment of the above aspects the inhibitor of insoluble aggregate formation is selected from pH, MgCl₂, propylene glycol, Pluronic-F68, Poloxamer 188, or ethanol. In an embodiment the inhibitor of insoluble aggregate formation is MgCl₂, wherein the concentration of MgCl₂ is from about 0.1 mM to about 300 mM, more preferably about 10 mM to about 300 mM, even more preferably about 30 mM to about 300 mM. In another embodiment the inhibitor of insoluble aggregate formation is propylene glycol, wherein the concentration of propylene glycol is from about 0.01% to about 10% (v/v), more preferably about 1% to about 10%. In another embodiment the inhibitor of insoluble aggregate formation is Pluronic-F68, wherein the concentration of Pluronic-F68 is from about 0.01% to about 5% (v/v), more preferably about 0.1% to about 1%. In another embodiment the inhibitor of insoluble aggregate formation is ethanol, wherein the concentration of ethanol is from about 0.01% to about 10% (v/v), more preferably about 0.1% to about 10%, even more preferably 0.1% to about 3%. In another embodiment the inhibitor of insoluble aggregate formation is pH, wherein the pH is maintained from about ±1.0 pH units or more from the isoelectric point (pI) of the protein in the formulation. More preferably the pH is maintained from about ±2.0 pH units or more from the isoelectric point (pI).

In another aspect, the invention provides methods for stabilizing a protein formulation against aggregate formation induced by one or more freeze/thaw cycles. In this aspect, the method of the invention comprises selecting a buffer system prior to the at least one freeze/thaw cycle; contacting the buffer system of with an amount of an inhibitor of insoluble aggregate formation effective to inhibit insoluble aggregate formation, prior to the at least one freeze/thaw cycle; and contacting the buffer system and inhibitor of insoluble aggregate formation of with an amount of a protein or protein fragment, prior to the at least one freeze/thaw cycle. In other embodiments of this aspect, the method can comprise the contacting with an amount of an inhibitor of insoluble aggregate formation prior to, during, or after the freeze/thaw cycle. Thus, the order of addition to the formulation can be interchanged, however the protein of interest must be in solution prior to the beginning of the freeze/thaw cycle(s).

In a further aspect, the invention provides methods for inhibiting protein aggregate formation in a protein solution that is subjected to one or more freeze/thaw cycles comprising selecting a buffer system, prior to the at least one freeze/thaw cycle; contacting the buffer system with an amount of an inhibitor of insoluble aggregate formation effective to inhibit insoluble aggregate formation, prior to the at least one freeze/thaw cycle; and contacting the buffer system and inhibitor of insoluble aggregate formation with an amount of a protein or protein fragment, prior to the at least one freeze/thaw cycle. As with the previously described aspect, certain embodiments of this method can comprise the contacting with an amount of an inhibitor of insoluble aggregate formation prior to, during, or after the freeze/thaw cycle(s).

In another aspect, the invention provides methods for stabilizing a protein formulation against aggregate formation induced by induced by agitation stress. In this aspect, the method of the invention comprises selecting a buffer system prior to the application (or threat/chance of) agitation stress; contacting the buffer system of with an amount of an inhibitor of insoluble aggregate formation effective to inhibit insoluble aggregate formation, prior to the agitation stress; and contacting the buffer system and inhibitor of insoluble aggregate formation of with an amount of a protein or protein fragment, prior to the application, threat, or chance of agitation stress. In other embodiments of this aspect, the method can comprise the contacting with an amount of an inhibitor of insoluble aggregate formation prior to, during, or after the agitation stress. Thus, the order of addition to the formulation can be interchanged, however the protein of interest must be in solution prior to the beginning of the agitation stress(es).

In a further aspect, the invention provides methods for inhibiting protein aggregate formation in a protein solution that is subjected to one or more physical agitation stresses comprising selecting a buffer system, prior to the agitation stress; contacting the buffer system with an amount of an inhibitor of insoluble aggregate formation effective to inhibit insoluble aggregate formation, prior to the agitation stress; and contacting the buffer system and inhibitor of insoluble aggregate formation with an amount of a protein or protein fragment, prior to the agitation stress. As with the previously described aspect, certain embodiments of this method can comprise the contacting with an amount of an inhibitor of insoluble aggregate formation prior to, during, or after physical agitation stress(es).

The invention also encompasses formulations comprising pharmaceutically effective amounts of protein together with suitable diluents, adjuvants and/or carriers. Other pharmaceutically acceptable excipients well known to those skilled in the art may also form a part of the subject compositions. These include, for example, various bulking agents, additional buffering agents, chelating agents, antioxidants, preservatives, cosolvents, and the like; specific examples of these could include, trimethylamine salts (“Tris buffer”), and EDTA. In one embodiment, more than one type of protein are included in the formulation. In another embodiment, no proteins other than the one protein of interest are part of the formulation.

Suitable pH ranges for the preparation of the formulations will depend on the particular protein or protein fragment of interest. It is particularly advantageous to select a buffer with a pH range that retains its buffering capacity in a range greater than or equal to 1 pH unit larger or smaller than the isoelectric point (pI) of the protein of interest. More preferably, the pH of the buffer system is stable in a range greater than or equal to 2 pH units larger or smaller than the pI of the protein. Further, it is particularly advantageous to select a buffer system that maintains pH over a large range of temperatures, particularly from about −80° C. to about 25° C. That is, the pH of the buffer system is preferably not significantly temperature dependent or responsive. In one embodiment the buffer is a potassium phosphate/potassium acetate mixed buffer system, having a pH range of about 4 to about 8, and a concentration range of about 1 mM to about 300 mM.

“Protein aggregate” or “protein aggregation” as used herein is taken to mean protein that is no longer in solution. While protein aggregate can mean agglomeration or oligomerization of two or more individual protein molecules, it is not limited to such a definition. Protein aggregates, as used in the art, can be soluble or insoluble; however for the purposes of the invention, protein aggregates are usually considered to be insoluble, unless otherwise specifically noted. Insoluble aggregates whose formation should be prevented in the process according to the invention are essentially understood as protein aggregates having a size of usually at least 1 μm but can also be in the range above 10 μm. The particles can be determined by suitable particle counting methods using commercial particle counting instruments such as, for example, the particle counting instrument AccuSizer 700 from PSS (Particle Sizing Systems, USA) or a Pacific Scientific HIAC Royco liquid particle counting system, model 9703, equipped with a LD400 laser counter. According to the USP (US-Pharmacopoeia) a maximum of 6000 particles in the range above 10 μm and a maximum of 600 particles in the range above 25 μm are allowed per injected dose of a pharmaceutical preparation. This can be achieved according to the invention in a simple manner for therapeutic compositions of proteins.

In accordance with this invention any protein can be utilized. Certain aspects of the invention are based on the use of the aqueous buffered solution and inhibitor of protein aggregate formation as recited in certain of the claims, and should not be interpreted as being limited by the specific protein dissolved therein.

The formulations are prepared in general by combining the components using generally available pharmaceutical combining techniques, known per se. A particular method for preparing a pharmaceutical formulation hereof comprises employing the protein purified according to any standard protein purification scheme, as well as those disclosed in the patents and patent applications describing antibodies A-E.

### EXAMPLES

The various antibodies used in the Examples are described in detail elsewhere in U.S. Pat. Nos. and U.S. patent application Ser. Nos.: 10/180,648 (Antibody A); 10/891,658 (Antibody B); 5,789,554, 6,254,868, 09/038,955, 09/590,284, 10/153,882 (Antibody C); 60/638,961 (Antibody D); 6,235,883 (Antibody E), all incorporated herein by reference.

Materials: CHO-derived antibodies were expressed and purified. The antibody was dialyzed extensively against distilled and deionized water and concentrated to ˜30 mg/mL. Due to the buffer range required for the Examples (pH 4-8), a combination of potassium phosphate and potassium acetate buffers was used. Potassium-based buffers were selected because of their frozen pH stability relative to sodium-based buffers. Potassium phosphate (K/PO₄), mono- and dibasic, and potassium acetate (K/OAc) were purchased from Mallinckrodt. Magnesium chloride (MgCl₂) hexahydrate was purchased from EM Science (Gibbstown, N.J.). Pluronic-F68 (Poloxamer) was purchased from Sigma. Ethanol (EtOH) and 1,2-propanediol (propylene glycol) were purchased from Aldrich Chemical Co.

### Example 1

Formulation Preparation

A series of formulations was prepared for each of the tested agents that inhibit freeze/thaw-inducted aggregate formation. Each formulation was prepared similarly. Test samples (2 mL) were prepared in 5 mL vials equipped with Daikyo stoppers. Concentrated buffer stock (20 mM K/OAc, 20 mM K/PO₄ at each tested pH value) was added to each sample to a final concentration of 5 mM K/OAc, 5 mM K/PO₄, at each pH value tested. Individual protein stock solutions (˜30 mg/mL) were added to each formulation to a final protein concentration of ˜10 mg/mL. Additional stock solutions of the agents that inhibit aggregate formation that were prepared include 5.0 M MgCl₂; 5% Pluronic-F68; 100% (v/v) EtOH; and 100% (v/v) propylene glycol. These stock solutions were added to the formulations to final concentration ranges noted in the disclosure below, typically 30-300 mM (MgCl₂); 0.01-1.0% (Pluronic-F68); 0.2-10% (EtOH); and 1-10% (propylene glycol). If necessary, deionized water was added to make final volume.

Freeze/Thaw Procedure

After preparing each formulation, the sample vials were sealed with stoppers and placed in a 5 cc×16 box with the appropriate vial spacer insert. The box was gently swirled to promote thorough, gentle mixing of the samples. After mixing, the samples were placed in a freezer (−80° C.) overnight. The following morning, the samples were removed from the freezer and placed at ambient (room 20-23° C.) temperature, allowing them to thaw. After the samples were completely thawed and equilibrated to ambient temperature, the samples, while in the box, were again mixed by gentle swirling. This freeze/thaw process was repeated for a total of 3 cycles.

Sample Analysis

After the 3 freeze/thaw cycles were completed, an initial visual examination of insoluble aggregate formation of the samples was performed. Thereafter, the insoluble aggregates were counted using a Pacific Scientific HIAC Royco liquid particle counting system, model 9703, equipped with a LD400 laser counter. Total assessment of the insoluble aggregate was quantified using the ≧2 μm detection limit. The detection limit of the instrument is approximately 18,000 counts/mL. If it appeared that heavy precipitation/aggregate formation occurred, the sample was diluted (typically 1:25 dilution) in order to quantify aggregate formation more accurately and avoid the instrument limitations.

Results

A. Dependence of Insoluble Aggregate Formation on pH.

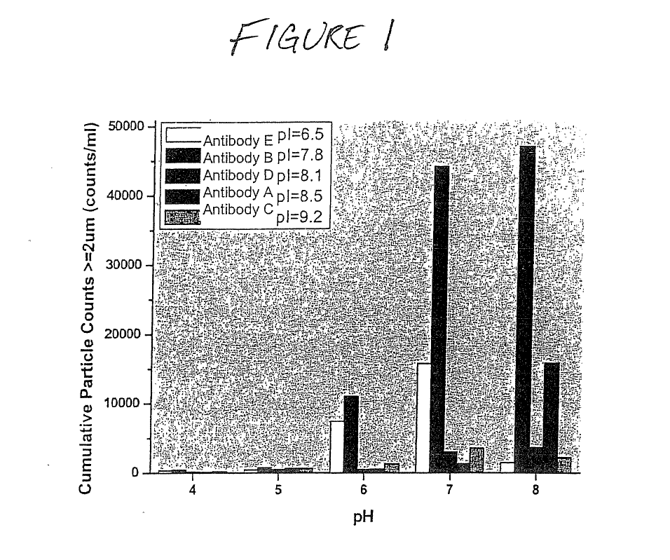

A general trend is observed for insoluble aggregation and its pH dependence between all IgG's tested. For all IgG's tested, pH values of between 4.0-5.0 gave consistently low particle counts for insoluble aggregate formation. From pH 6.0-8.0, the counts of insoluble aggregates were highly dependent on the isoelectric point (pI) of the specific protein. As is seen for antibody A, antibody C, and antibody D (pI values of 8.5, 9.2, and 8.7, respectively), total particle counts were considerably lower (<1500) when compared to antibody B, and antibody E (pI values of 7.8 and 6.5, respectively). Total particle counts for antibody B and antibody E at pH 6.0 are ˜11,000 and ˜7,400, respectively. Antibody B has an unusually high level of insoluble aggregates as the pH approaches the pI of the protein. Antibody C and antibody D appear to be slightly resistant to forming insoluble aggregates during freeze/thaw and changes in pH most likely due to the pH range tested. These two proteins have pI's of 9.2 and 8.7, which are the highest pI of all the proteins tested in this work (FIG. 1). These trends indicate that to inhibit insoluble aggregate formation, buffer pH ranges should be determined by the pI of the particular protein in a formulation. Ideally, the pH of the buffer system should be at least a full pH unit higher or lower than the pI value of the protein.

B. Dependence of Insoluble Aggregate Formation on Magnesium Chloride.

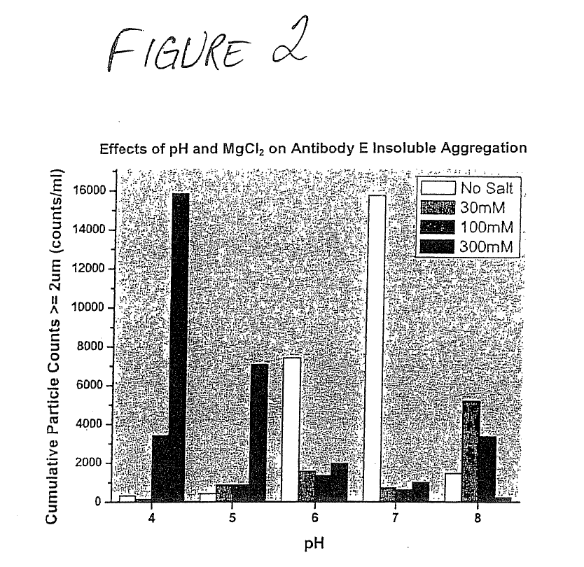

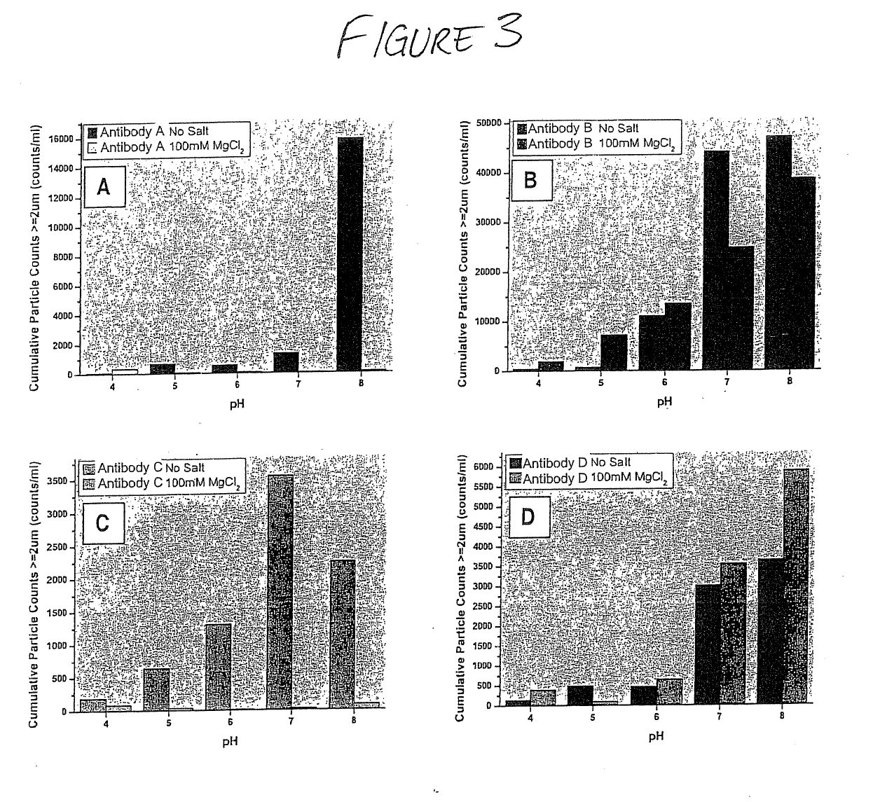

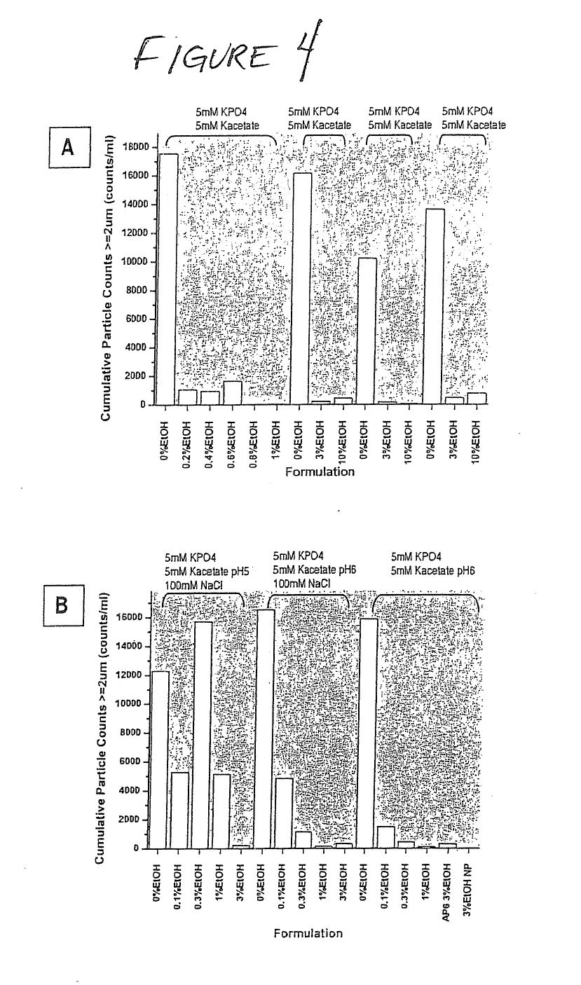

Using the pH screen described in (A) above for comparison, the addition of between 30-300 mM MgCl₂ can suppress insoluble aggregate formation induced by three cycles of freeze/thaw. The conditions that produce the most insoluble aggregates in antibody E formulations are significantly suppressed with the introduction of MgCl₂. This is most prominently seen between the pH range of 6-7. FIG. 2 shows suppression of insoluble aggregates between 30-300 mM MgCl₂ for antibody E only. FIG. 3 shows the effect of MgCl₂ on insoluble aggregation on antibodies A-D at 100 mM MgCl₂ concentration. Suppression of insoluble aggregates by MgCl₂ is a generally observed phenomenon in all proteins except for antibody D. Antibody A is a well-behaved protein during freeze/thaw. Insoluble aggregates are slight in most conditions tested, except for pH 8. This is likely due to the fact that pH 8 is close to the pI of antibody A (8.5) and contains significant insoluble aggregates (˜16,000 counts/mL). The inclusion of MgCl₂ at pH 8 for antibody A significantly reduces the insoluble aggregate count to <50 counts/mL. Antibody B has the least amount of protection against insoluble aggregate formation after addition of MgCl₂. Under all conditions, addition of MgCl₂ either contains less insoluble aggregates when compared to just buffered solution or an equivalent amount of aggregate for antibody B. Antibody D appears to be an exception to this observation. The addition of MgCl₂ in the formulation either maintains the level of insoluble aggregates when compared to buffer alone, or increases the number of insoluble aggregates in pH range 7-8.

C. Dependence of Insoluble Aggregate Formation on ethanol

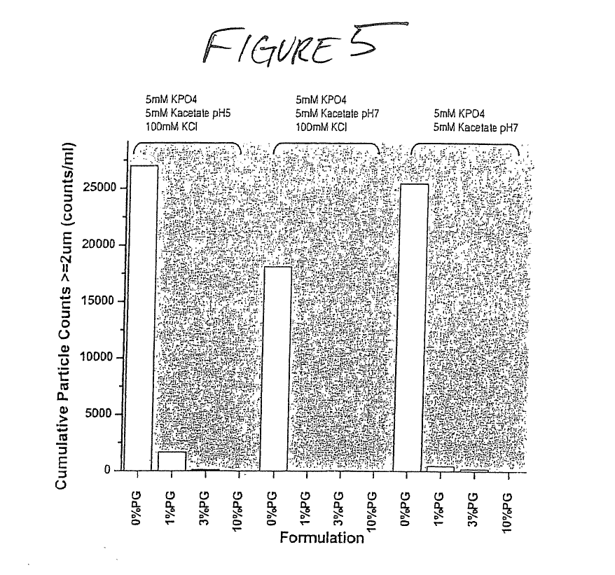

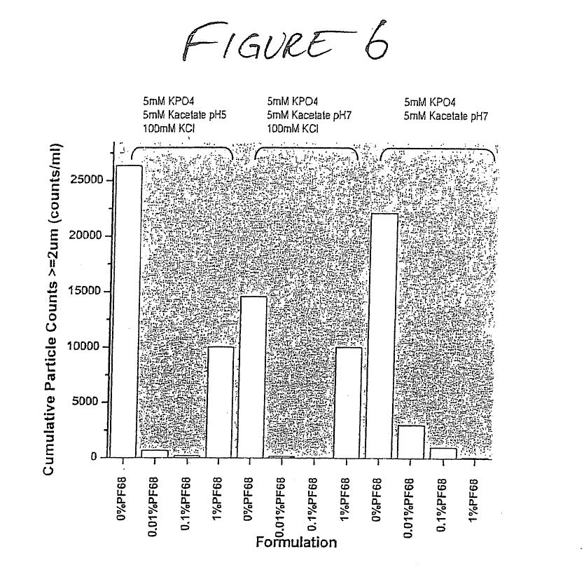

Using previous conditions known to generate high amounts of insoluble aggregates with antibody E, the addition of low concentrations of ethanol decreases the number of insoluble aggregates. These insoluble aggregate-forming buffers are: 5 mM K/PO₄, 5 mM K/OAc, with or without potassium or sodium chloride (100 mM KCl, at pH 5.0 or 7.0; 100 mM NaCl, at pH 5 or 6). Three freeze/thaw cycles of antibody E in the above buffer conditions induces aggregate formation of about 15,000 counts/mL. Under the same conditions the addition of ethanol (at 0.1% (v/v)) reduced the amount of insoluble aggregate formation by more than 50%. Addition of 0.2% (v/v) ethanol decreases the amount of insoluble aggregate by nearly two orders of magnitude. Ethanol added in amounts of 0.8-10% (v/v) nearly eliminates insoluble aggregates induced by three cycles of freeze/thaw. FIG. 4 illustrates the effects of ethanol on insoluble aggregate formation for antibody E in (A) KCl- and (B) NaCl-containing buffer systems.

D. Dependence of Insoluble Aggregate Formation on Propylene Glycol.

Using already described conditions for maximal insoluble aggregate formation (above (C)), the addition of various amounts of propylene glycol reduces precipitation of antibody E. In all conditions tested (5 mM K/PO4, 5 mM KOAc, +/− 100 mM KCl, pH 5 or 7), the addition of 1% propylene glycol reduced the insoluble aggregate amount by ˜1.5 orders of magnitude. Further increase in propylene glycol amounts reduced the level of precipitation (>2 orders of magnitude). FIG. 5 illustrates the inhibitory effects that propylene glycol has on insoluble aggregate formation in destabilizing buffer systems.

E. Dependence of Insoluble Aggregate Formation on emulsifying/wetting agent.

Poloxamer 188 and Pluronic-F68 are classified as fat emulsifiers and wetting agents when present in concentration ranges of 0.01-5% (Rowe, et al., Handbook of Pharmaceutical Excipients, 4^{th} Ed., Weller, P. J. (ed.); Pharmaceutical Press (London) and American Pharmaceutical Association (Washington D.C.), 2003. pp. 447-449). Using the destabilizing buffer system described above (C, D), Pluronic-F68 was added to a concentration of 0.01-1%. Addition of Pluronic-F68 in this concentration range inhibited the formation of insoluble aggregate formation (FIG. 6).

### Example 2

Inhibition of Protein Aggregate Formation During Agitation Stress

Each formulation is prepared using the antibodies as described in Example 1, with buffer conditions including: (a) 5 mM sodium acetate, 5 mM potassium phosphate, pH 7 (control sample); (b) 5 mM sodium acetate, 5 mM potassium phosphate, 100 mM MgCl₂, pH 7; (c) 5 mM sodium acetate, 5 mM potassium phosphate, 0.1% Pluronic F68, pH 7; and (d) 5 mM sodium acetate, 5 mM potassium phosphate, 10% propylene glycol, pH 7. These formulations are prepared using any method known to those skilled in the art, such as dialysis, diafiltration, buffer exchange (chromatography, centrifuge filtration, etc.). Those of skill in the art are able to identify the proper materials needed for such preparation (molecular weight cut-off of dialysis tubing and diafiltration membranes, etc.). Once a typical protein concentration is achieved (e.g., ˜10 mg/mL), the sample vials are sealed with stoppers and placed in a 5 cc×16 box with the appropriate vial spacer insert. The box is gently swirled to promote and ensure thorough, gentle mixing of the samples.

After mixing, the samples are subjected to shipping stimulation (12 hours ground and 12 hours air vibrations that are representative of a truck and airplane). If shipping stimulation is not available, simulated shipping conditions can be achieved through a variety of ways, such as on an orbital shaker (e.g., VWR OS-500 orbital shaker) operating at 500 rpm for 72 hours or longer (VWR OS-500 orbital shaker).

Sample Analysis

After the agitation stress is completed, an initial visual examination of insoluble aggregate formation of the samples is performed. Thereafter, any insoluble aggregates are counted using a Pacific Scientific HIAC Royco liquid particle counting system, model 9703, equipped with a LD400 laser counter. Total assessment of the insoluble aggregate is quantified using the ≧2 μm detection limit. The detection limit of the instrument is approximately 18,000 counts/mL. If it appears that heavy precipitation/aggregate formation occurred, the sample is diluted (typically 1:25 dilution) in order to quantify aggregate formation more accurately and avoid the instrument limitations.

While the invention is described in particular aspects and embodiments, the foregoing description and Examples should not be interpreted as limiting the invention. The invention covers various modifications and equivalent formulations apparent to those of skill in the art, and included within the spirit and scope of the appended claims.

What is claimed:

1. A formulation comprising:
a) a pharmaceutically acceptable amount of an antibody selected from the group consisting of antibody A, antibody B, antibody C, antibody D, antibody E, or fragments thereof;
b) a buffer; and
c) an inhibitor of insoluble aggregate formation.

2. The formulation of claim 1, wherein the buffer has a pH range of about 4.0 to about 8.0.

3. The formulation of claim 1, wherein the inhibitor of insoluble aggregate formation is at least one of MgCl₂, propylene glycol, Pluronic-F68, Poloxamer 188, ethanol, or combinations thereof.

4. The formulation of claim 2, wherein the buffer is a phosphate buffer.

5. The formulation of claim 3, wherein the inhibitor of insoluble aggregate formation is MgCl₂.

6. The formulation of claim 5, wherein the amount of MgCl₂ is about 0.1 mM to about 300 mM.

7. The formulation of claim 3, wherein the inhibitor of insoluble aggregate formation is propylene glycol.

8. The formulation of claim 7, wherein the amount of propylene glycol is about 0.01% to about 10% (v/v).

9. The formulation of claim 3, wherein the inhibitor of insoluble aggregate formation is Pluronic-F68.

10. The formulation of claim 9, wherein the amount of Pluronic-F68 is about 0.01% to about 5% (v/v).

11. The formulation of claim 3, wherein the inhibitor of insoluble aggregate formation is Poloxamer 188.

12. The formulation of claim 11, wherein the amount of Poloxamer 188 is about 0.01% to about 5% (v/v).

13. The formulation of claim 3, wherein the inhibitor of insoluble aggregate formation is ethanol.

14. The formulation of claim 13, wherein the amount of ethanol is about 0.01% to about 10% (v/v).

15. A method for stabilizing a protein formulation against aggregate formation induced by one or more freeze/thaw cycles comprising:
(a) selecting a buffer system, prior to the at least one freeze/thaw cycle;
(b) contacting the buffer system of (a) with an amount of an inhibitor of insoluble aggregate formation effective to inhibit insoluble aggregate formation, prior to the at least one freeze/thaw cycle; and
(c) contacting the buffer system and inhibitor of insoluble aggregate formation of (b), with an amount of a protein or protein fragment, prior to the at least one freeze/thaw cycle.

16. A method for inhibiting protein aggregate formation in a protein solution that is subjected to one or more freeze/thaw cycles comprising:
(a) selecting a buffer system, prior to the at least one freeze/thaw cycle;
(b) contacting the buffer system of (a) with an amount of an inhibitor of insoluble aggregate formation effective to inhibit insoluble aggregate formation, prior to the at least one freeze/thaw cycle; and
(c) contacting the buffer system and inhibitor of insoluble aggregate formation of (b), with an amount of a protein or protein fragment, prior to the at least one freeze/thaw cycle.

17. A method for inhibiting protein aggregate formation induced by one or more freeze/thaw cycles comprising contacting a solution comprising a protein or protein fragment with an amount of an inhibitor of insoluble aggregate formation prior to, during, or after the at least one freeze/thaw cycle.

18. A method for preparing a protein formulation stabilized against protein aggregate formation induced by one or more freeze/thaw cycles comprising:
(a) selecting a buffer system;
(b) contacting the buffer system of (a) with an amount of an inhibitor of insoluble aggregate formation effective to inhibit insoluble aggregate formation; and
(c) contacting the buffer system and inhibitor of insoluble aggregate formation of (b), with an amount of a protein or protein fragment, prior to the at least one freeze/thaw cycle.

19. A protein formulation having increased stability against insoluble aggregate formation induced by one or more freeze/thaw cycles, comprising:
a) a protein or protein fragment;
b) an amount effective to inhibit insoluble aggregate formation of an inhibitor of insoluble aggregate formation selected from MgCl₂, propylene glycol, Pluronic-F68, Poloxamer 188, or ethanol; and
c) a buffer system.

20. The protein formulation of claim 19, wherein the buffer system is selected based on the isoelectric point (pI) of the protein or protein fragment of (a).

21. The protein formulation of claim 20, wherein the buffer system is equal to or greater than 2 pH units higher or lower than the pI of the protein or protein fragment of (a).

22. The formulation of claim 1, wherein the buffer has a pH greater than 2 pH units higher or lower than the isoelectric point of the antibody of (a).

23. The formulation of any of claims 1-3, wherein the antibody is antibody E.

24. A method for stabilizing a protein formulation against aggregate formation induced by agitation stress comprising:
(a) selecting a buffer system, prior to the agitation stress;
(b) contacting the buffer system of (a) with an amount of an inhibitor of insoluble aggregate formation effective to inhibit insoluble aggregate formation, prior to the agitation stress; and
(c) contacting the buffer system and inhibitor of insoluble aggregate formation of (b), with an amount of a protein or protein fragment, prior to the agitation stress.

25. A method for inhibiting protein aggregate formation in a protein solution that is subjected to agitation stress comprising:
(a) selecting a buffer system, prior to the agitation stress;
(b) contacting the buffer system of (a) with an amount of an inhibitor of insoluble aggregate formation effective to inhibit insoluble aggregate formation, prior to the agitation stress; and
(c) contacting the buffer system and inhibitor of insoluble aggregate formation of (b), with an amount of a protein or protein fragment, prior to the agitation stress.

26. A method for inhibiting protein aggregate formation induced by agitation stress comprising contacting a solution comprising a protein or protein fragment with an amount of an inhibitor of insoluble aggregate formation prior to, during, or after the agitation stress.

27. A method for preparing a protein formulation stabilized against protein aggregate formation induced by agitation stress comprising:
(a) selecting a buffer system;
(b) contacting the buffer system of (a) with an amount of an inhibitor of insoluble aggregate formation effective to inhibit insoluble aggregate formation; and
(c) contacting the buffer system and inhibitor of insoluble aggregate formation of (b), with an amount of a protein or protein fragment, prior to the agitation stress.

28. A protein formulation having increased stability against insoluble aggregate formation induced by agitation stress, comprising:
a) a protein or protein fragment;
b) an amount effective to inhibit insoluble aggregate formation of an inhibitor of insoluble aggregate formation selected from MgCl₂, propylene glycol, Pluronic-F68, Poloxamer 188, or ethanol; and
c) a buffer system.

29. The protein formulation of claim 28, wherein the buffer system is selected based on the isoelectric point (pI) of the protein or protein fragment of (a).

30. The protein formulation of claim 29, wherein the buffer system is equal to or greater than 2 pH units higher or lower than the pI of the protein or protein fragment of (a).

31. The formulation of claim 28, wherein the agitation stress is applied to the sample during shipping on land or sea, or in the air.
